(19) United States
(12) Patent Application Publication
(10) Pub. No.: US 2010/0189601 A1
(43) Pub. Date: Jul. 29, 2010

(54) CAPILLARY

(76) Inventors: Anne Marie Crawford, Sittingbourne (GB); Austin John Kirk, Sittingbourne (GB)

Correspondence Address: WOOD, PHILLIPS, KATZ, CLARK & MORTIMER, 500 W. MADISON STREET, SUITE 3800, CHICAGO, IL, 60661, US

(21) Appl. No.: 12/532,055

(22) PCT Filed: Mar. 20, 2008
(86) PCT No.: PCT/GB2008/050207

§ 371 Date: Mar. 12, 2010

(30) Foreign Application Priority Data
Mar. 21, 2007 (GB) 0705418.2

(51) Int. Cl. G01N 33/53; B01L 3/00

(52) U.S. Cl. 422/69; 422/100

(57) ABSTRACT

This invention relates to a capillary channel comprising a first pair of opposing walls defining a width and a second pair of opposing walls defining a depth, wherein the channel has an aspect ratio of 10-100 defined as the ratio of the width to the depth of the channel and wherein an internal surface of at least one of the second pair of opposing walls is roughened. The capillary channel is preferably incorporated into a sensor.

This invention relates to a capillary, and in particular to a capillary channel adapted for improved flow.

The use of small channels in which liquid flow is controlled by capillary flow forces is becoming more common in in vitro diagnostic devices (IVD). Channels of only a few tens to a few hundreds of micrometres in size mean sample and reagent volumes can be minimised, often to a few microlitres (μL) thereby reducing cost, instrument complexity and test times. As a result, manufacturability is simplified which offers increased margins and excellent repeatability, both of which are important since the marketplace primarily demands single-use devices. Such devices are ideally suited to use by non-specialist operators in near-patient and “point-of-care” (PoC) applications especially where the chemistry involves the use of antigen/antibody reactions in an immunoassay format.

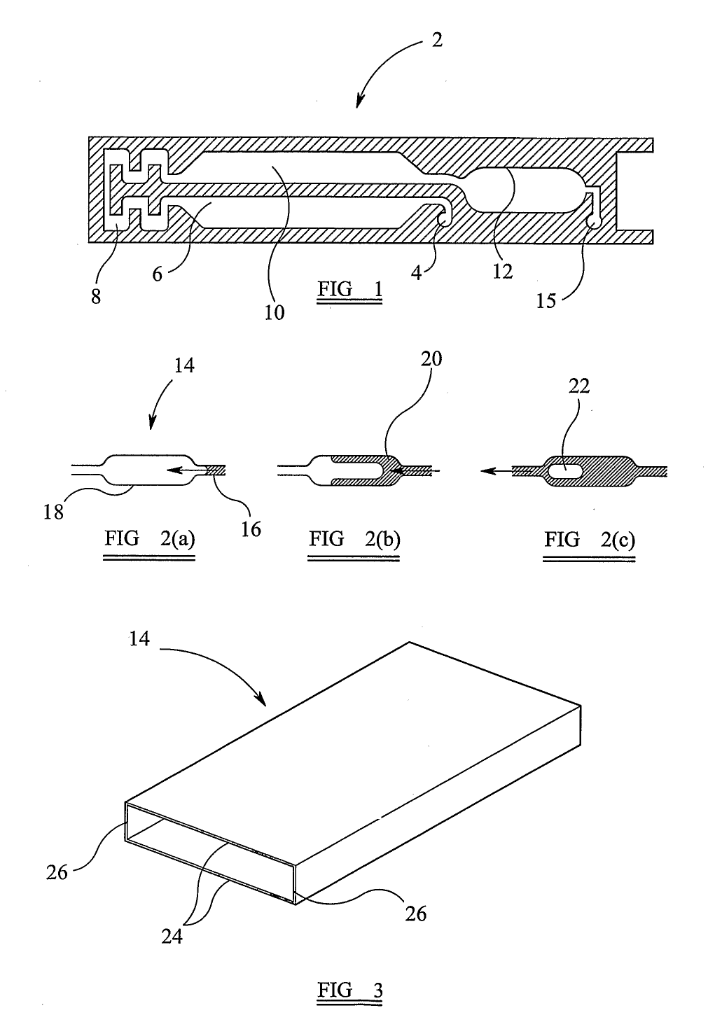

In a typical device 2 as shown in FIG. 1, a fluid sample, such as a sample of biological fluid, e.g. blood, is introduced into the device 2 at a sample inlet 4. The fluid sample is drawn into a first reagent microchannel 6 by capillary forces and subsequently caused to move in order to mix with liquid and/or solid reagents, for example in a mixing labyrinth 8, before finally being moved via a second reagent microchannel 10 to a sensor area 12 of the device 2. Movement can be achieved, for example, by air flow (pressure or vacuum), by hydraulic movement using a “finger pump”, or by electrical or electrostatic means. The mixing labyrinth 8 is not essential but is included to speed up mixing which can be achieved, albeit less efficiently, by passing the materials to be mixed through a simple restricted orifice.

Previously, the most common method of fabrication of such disposable devices was by injection moulding. Increasingly, the preferred manufacturing method is lamination of suitably shaped or die-cut sheeted materials with pressure sensitive adhesives (PSAs) to form linear channels a few millimetres in width and tens to hundreds of micrometres deep. One problem with such channels where the aspect ratio (the ratio of the width to the depth) is in the range 10 to 100 is that movement of fluid back-and-forth, for example to encourage mixing of a dried-down reagent, and the multiple drying and re-wetting of the surface that ensues, tends to form bubbles or air-filled voids that may deleteriously interfere with the signal generated when the sample/reagent mixture is moved to the sensor area.

This bubble formation is frequently the result of differences in hydrophobicity and hydrophilicity of the surfaces forming the channels. FIG. 2 shows a capillary channel 14 having a first portion 16 and a second portion 18, in which the second portion 18 is wider than the first portion 16. Bubble formation may occur as the fluid sample 20 enters the capillary channel. At point (a) the fluid enters a wider portion of the capillary channel and at point (b) the fluid forms a meniscus. As the fluid moves along the capillary channel, contact between the fluid and the wall of the capillary channel increases on account of the shape of the channel and variations in the surface energy leading to unwanted bubble 22 formation at point (c).

Thus, in a rectangular capillary, under circumstances where the edges of the channel are linear the capillary force at the edges appears significantly greater than in the centre of the channel. This encourages the liquid to “chase” up the edges far ahead of the bulk of the liquid, causing the formation of bubbles in the centre of the channel.

This bubble formation can, to some extent, be mitigated by coating the surfaces involved with suitable chemicals to counteract the enhanced capillary action that occurs at the edges of a rectangular capillary, evening-out the “wetability” of the surfaces involved and the liquid flow. This, however, introduces another step or steps into the manufacture of the device, increasing cost and complexity, and the materials involved in changing the properties of the surfaces can interfere with the composition of the fluids and subsequent analyte detection dynamics, especially when they re-dissolve in the fluid passing over them.

Alternatively, some IVD developers have attempted to improve the wetability of the surface by changing the surface morphology to encourage capillary action at the micro level, for example by adding micrometre-sized pillars, peaks or steps. See US 2005/0136552 for an example of this methodology. The addition of such roughened surfaces is readily achievable by, for example, micromachining of mould tools where the components are injection moulded.

However, introducing a roughened surface is much harder to achieve if the disposable device is fabricated from die-cut sheeted material, without employing a complex multi-step thermoformed or embossed pre-treatment. Such complex multi-step methods are prohibitive in terms of cost in disposable laminated devices. There remains a requirement in the art, therefore, for a solution to the problem of bubble formation in a capillary channel formed as a laminated structure.

Accordingly, the present invention provides a capillary channel comprising a first pair of opposing walls defining a width and a second pair of opposing walls defining a depth, wherein the channel has an aspect ratio of 10-100 defined as the ratio of the width to the depth of the channel and wherein an internal surface of at least one of the second pair of opposing walls is roughened.

The present invention will now be described with reference to the accompanying drawings, in which:

FIG. 1 shows a sensor incorporating capillary channels according to the prior art;

FIG. 2 shows a conventional capillary channel;

FIG. 3 shows a capillary channel in which the width is greater than the depth according to the present invention;

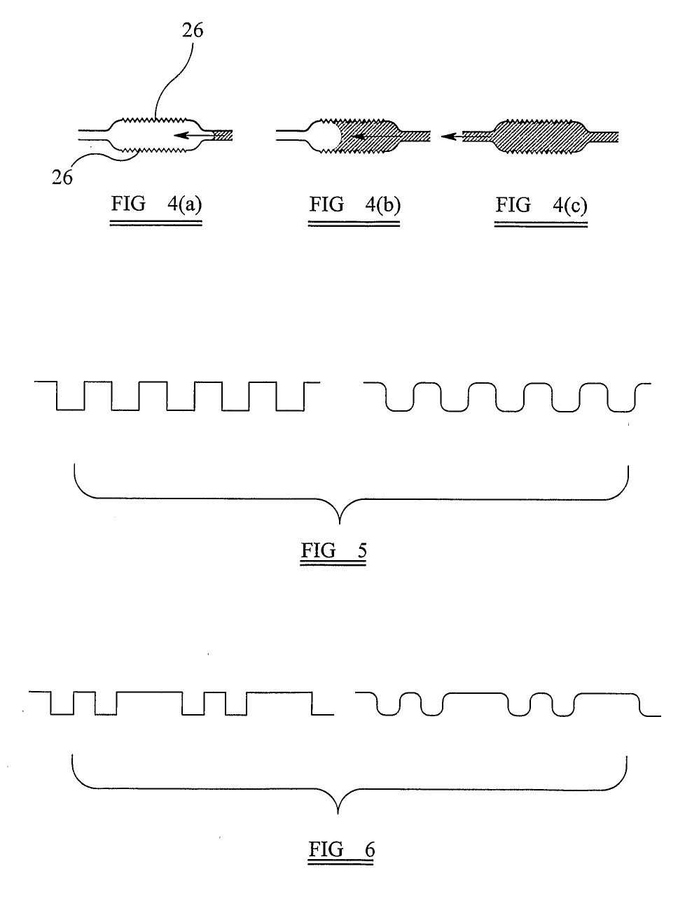

FIG. 4 shows a capillary channel of the present invention;

FIGS. 5-7 show discontinuities in the wall of capillary channels according to the present invention; and

FIG. 8 shows a sensor incorporating a capillary channel of the present invention.

FIG. 3 shows a capillary channel 14 according to the present invention. The capillary channel 14 comprises a first pair of opposing walls 24 defining a width and a second pair of opposing walls 26 defining a depth, wherein the width is greater than the depth. FIG. 4 shows the capillary channel 14 of the present invention in cross section in which the internal surfaces of both of the second pair of opposing walls 26 is roughened. Either one or both of the second pair of opposing walls 26 may be roughened although, preferably, both are roughened. As the fluid sample is caused to move from point (a) via point (b) to point (c), the roughened surface minimises or prevents bubble formation.

In a typical device formed as a laminated structure, the channels 14 are cut into a spacer, for example die-cut into a plastics film layer. The spacer is typically has a thickness of 50-500 μm. Suitable materials include polyester (e.g. Mylar, Melinex) or polycarbonate (e.g. Lexan). The spacer is then laminated between two planar substrates (“lids”) formed of a similar material to the spacer using PSA to form the required flow path. Thus, in a preferred embodiment of the present invention, the capillary channel comprises a laminate structure wherein the first pair of opposing walls is formed from two planar substrates and the second pair of opposing walls is formed from channels cut into a spacer sandwiched between the two planar substrates.

The capillary channel of the present invention preferably has a width of 1-5 mm; the channel also preferably has a depth of 10-500 μm. The channel has a width which is greater than the depth and the channel has an aspect ratio of 10-100 defined as the ratio of the width to the depth of the channel.

It has been found that the flow in a capillary channel can be evened-out by roughening the surface of the second pair of opposing walls 26. Roughening may be achieved using techniques known in the art, for example adding small ridges, steps or “teeth” to the second pair of opposing walls 26, i.e. the die-cut edges of the PSA laminated spacers.

Surprisingly, the roughened surface retains small quantities of fluid and/or air when the bulk sample is moved through the channel which appears to encourage flow in the centre of the channel, minimising large bubble formation, when the bulk liquid is returned to the channel. It is surprising that the roughening of the narrower or shallower surfaces has the desired effect.

An advantage of the present invention is that the first pair of opposing walls does not need to be roughened and preferably, the internal surfaces of these walls are smooth. However, an internal surface of one or both of the first pair of opposing walls may also be roughened if desired.

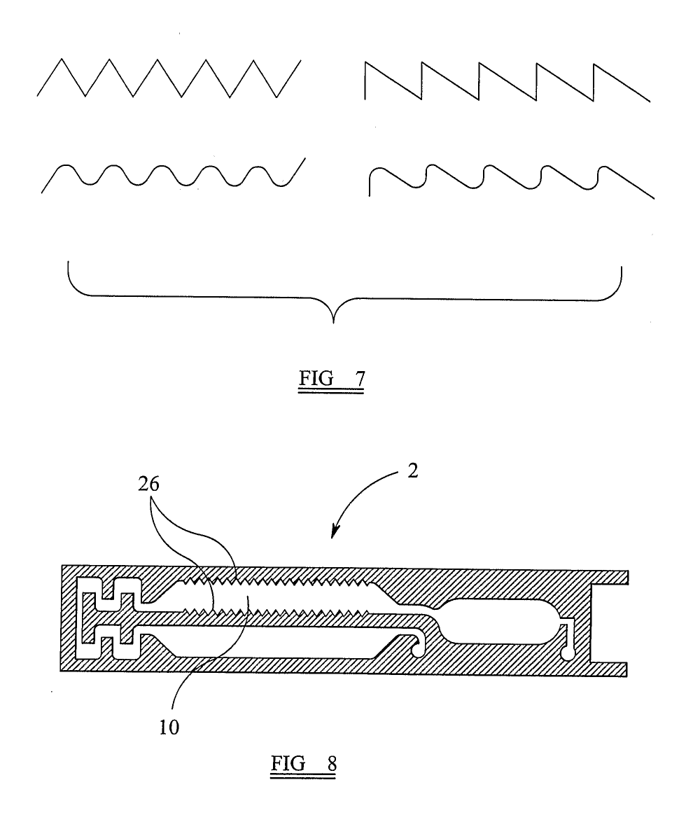

Roughening of the surface introduces one or more discontinuities into an otherwise smooth surface. The roughened surface may comprise square, rectangular, circular and/or triangular discontinuities. The discontinuities may be raised or depressed. The discontinuities tend to have a height (or depth) of about 1-2,000 μm. Preferably, the discontinuities repeat every 10-2,000 μm. Possible shapes of the roughened surface are shown in FIGS. 5, 6 and 7. FIG. 5 shows a symmetrical repeating pattern of square or rectangular shapes which preferably repeats every 10-2,000 μm. FIG. 6 shows an asymmetrical repeating pattern of square or rectangular shapes which preferably includes at least one square or rectangle every 10-2,000 μm. FIG. 7 shows a symmetrical repeating pattern of triangular shapes which may be upright triangles or “saw-tooth” in shape and which preferably repeats every 10-2,000 μm. The angular portions of the discontinuities, such as the tops of the saw-teeth or the inner angles at the base of the square-shaped discontinuities or notches, may be radiused (i.e. having small inner and outer curves rather than being “pointed” angles, like the corners of a triangle or square). Radiusing these corners will further improve the flow characteristics of the channels. Preferably the radiused angular portions have a radius of 0.1-1 mm.

Although a plurality of discontinuities is preferred, a single discontinuity (notch) is sufficient if it is placed near the bottleneck at the exit of chamber 14. More preferably, two discontinuities are placed opposite one another.

Without wishing to be bound by theory, the present invention is believed to work in four possible ways, some or all of which will contribute to the reliability of flow in any particular case:

Firstly, the roughened surface means that the fluid at the edges of the capillary channel has to travel farther, i.e. in and out of each discontinuity, rather than running straight up the edge, and this increased distance slows the fluid at the edge without slowing the fluid in the centre.

Secondly, the roughened surface reduces, but does not eliminate, the sample chasing up the spacer edges by interfering with the enhanced capillary action that is normally seen at the capillary walls. Thus bubble formation is discouraged in the mixing chamber. In practice, the roughened surfaces do not have to become filled in order to see their beneficial effect. Indeed, small quantities of air trapped in these notches breaks up the enhanced capillary action normally seen at the wall. Where fluid does chase up the edges during filling of the mixing chamber, when fluid movement ceases the centre portion of the fluid “slug” continues to move forward to meet the level of fluid at the edges. This effect is strong enough that sometimes the central fluid portion ends up in advance of the liquid at the edges providing a “convex meniscus” effect. This is likely to be the result of surface tension on the front edge of the fluid sample.

Thirdly, when the fluid flow is “back-and-forth”, it encourages the retention of small amounts of fluid between the discontinuities of the spacer evening-out the “wetability” of the edges of the channel.

Fourthly, when air bubbles do form they tend to become trapped at the (air filled) discontinuities and remain static during fluid movement. Thus they are discouraged from being transferred into the reading chamber with the liquid sample. They are presumably being driven to combine with air in the notches in order to minimise the surface area in contact with the liquid. Again, this is a surface tension effect. Air bubbles may be driven to displace the fluid from discontinuities and become inserted into them in order to present a smaller surface area to the fluid.

In a preferred embodiment, the capillary channel of the present invention is introduced in a sensor. FIG. 8 shows a sensor akin to the sensor shown in FIG. 1 but the sensor of FIG. 8 incorporates the capillary channel 14 of the present invention as the second reagent microchannel 10 in which the internal surfaces of the second pair of opposing walls 26 are roughened.

Suitable sensors which may incorporate the capillary channel 14 of the present invention are the sensors set out in WO 90/13017, WO 2004/090512 and WO 2006/079795.

Accordingly, the present invention also provides the use of the capillary channel as defined herein as a fluid-sample containment element in a sensor. The present invention also provides a sensor for detecting an analyte in a fluid sample, the sensor comprising a substrate, a reagent for binding the analyte, a radiation source for irradiating the reagent, a transducer having a pyroelectric or piezoelectric element which is capable of transducing energy generated by the reagent on irradiation into an electrical signal, electrodes in electronic communication with the transducer, and a processor which is capable of converting the electrical signal into an indication of the concentration of the analyte, wherein the substrate incorporates the capillary channel as described herein.

1. A capillary channel comprising a first pair of opposing walls defining a width and a second pair of opposing walls defining a depth, wherein the channel has an aspect ratio of 10-100 defined as the ratio of the width to the depth of the channel and wherein an internal surface of at least one of the second pair of opposing walls is roughened.

2. A capillary channel as claimed in claim 1, wherein an internal surface of both of the second pair of opposing walls is roughened.

3. A capillary channel as claimed in claim 1, wherein the first pair of opposing walls is not roughened.

4. A capillary channel as claimed in claim 1, wherein the channel has a width of 0.1-10 mm.

5. A capillary channel as claimed in claim 1, wherein the channel has a depth of 10-1000 μm.

6. A capillary channel as claimed in claim 1, comprising a laminate structure wherein the first pair of opposing walls is formed from two planar substrates and the second pair of opposing walls is formed from channels cut into a spacer sandwiched between the two planar substrates.

7. A capillary channel as claimed in claim 1, wherein the roughened surface comprises square, rectangular and/or triangular discontinuities.

8. A capillary channel as claimed in claim 7, wherein the discontinuities repeat every 10-5,000 μm.

9. A capillary channel as claimed in claim 7, wherein the discontinuities have a height of 1-2,000 μm.

10. A capillary channel as claimed in claims 7, wherein angular portions of the discontinuities are radiused.

11. A method of using the capillary channel as claimed in claim 1 as a fluid-sample containment element in a sensor.

12. A sensor for detecting an analyte in a fluid sample, wherein the sensor incorporates the capillary channel as claimed in claim 1.

13. A sensor as claimed in claim 12, wherein the sensor comprises a substrate, a reagent for binding the analyte, a radiation source for irradiating the reagent, a transducer having a pyroelectric or piezoelectric element which is capable of transducing energy generated by the reagent on irradiation into an electrical signal, electrodes in electronic communication with the transducer, and a processor which is capable of converting the electrical signal into an indication of the concentration of the analyte, wherein the substrate incorporates the capillary channel as claimed in claim 1.
